(19) United States
(12) Patent Application Publication
(10) Pub. No.: US 2005/0213036 A1
(43) Pub. Date: Sep. 29, 2005

(54) System and method for ocular input to an automation system

(75) Inventor: Carl P. Morse, Milford, NH (US)

Correspondence Address: SQUARE D COMPANY;INTELLECTUAL PROPERTY DEPARTMENT, 1415 SOUTH ROSELLE ROAD, PALATINE, IL, 60067, US

(73) Assignee: Schneider Automation Inc.

(21) Appl. No.: 10/465,105

(22) Filed: Jun. 19, 2003

(51) Int. Cl. A61B 3/14

(52) U.S. Cl. 351/209

(57) ABSTRACT

A system and method for providing hands free input to a subcomponent of an automation system. The system uses an ocular tracking device to allow a user of the system to provide an input signal to the subcomponent by movement of the user's eyes.

### RELATED APPLICATIONS

The present application is being filed concurrently with applications for “System And Method For Voice Input To An Automation System” (Attorney Docket No. SAA-95 (402P311)), serial number unknown at present; “System And Method For Voice Output From An Automation System” (Attorney Docket No. SAA-97 (402P313)), serial number unknown at present; and “Biometric PLC Access And Control System And Method” (Attorney Docket No. SAA-98 (402P314)), serial number unknown at present;, which are incorporated herein by reference. These applications have the same the inventor.

### TECHNICAL FIELD

The present invention is generally related to a hands free system and method for inputting data or controlling an automation system, and more particularly to a system and method for using ocular tracking systems to provide input for facilitating use of a machine or other subcomponent of an automation system.

### BACKGROUND OF THE INVENTION

Programmable logic controllers (PLCs) have been used for many years in factory settings to control the execution of numerous independent tasks of one or more devices or pieces of machinery. Such controllers can be configured to be compact, robust and capable of operating in harsh environments that generally deter using a personal computer (PC). In view of these and other attributes, PLCs are a popular choice for implementation in automation systems that are typically located in the factory settings.

PLCs operate in accordance with a stored control program that causes the controller to examine the state of the equipment under its control by evaluating signals from one or more sensing devices. Additionally, PLCs are typically configured to receive input from a user or operator through an IO module or a human-machine interface (HMI).

There are many instances in the factory setting where a user or operator of a device or machine located in the factory, is required to input certain information or control signals during operation of the device or machine. Moreover, this often occurs in circumstances where it is difficult or impossible for the operator to utilize the traditional input system (e.g., a control panel, keyboard or touch sensitive screen) which may require physically pushing a button or flipping a switch. For example, this situation may occur where an operator is utilizing a portion of a machine remote from the control center of the machine, or is required to hold a part in place during operation of the machine. In such circumstances, it becomes necessary for two operators to be present (one to perform the remote task, and one to provide the required input at the control center) to effectively operate the machine. This increases the production costs, which are either passed to the consumer or result in lower profitability.

### SUMMARY OF THE INVENTION

The present invention provides a hands free system and method for inputting data or control signals into a device or machine. The system and method are preferably implemented in connection with a device or machine which form a part or subcomponent of an automation system. Specifically, the hands free system and method involve utilizing ocular tracking systems to input data or control information to the device or machine. This allows an operator of the machine to provide data or control the machine from a location remote from any control system for the machine. The invention provides for using a PLC process to interpret the location where a user is looking and determine an action to be implemented by the process.

The present invention could be applicable to provide an ocular controlled input as a direct input to a PLC operatively associated with a device or machine. Alternatively, the ocular controlled input can be provided through an 10 module, or a Human Machine Interface connected to the automation system. The invention combines ocular tracking technology, such as that used for controlling movement of a cursor on a computer screen or display, with an industrial automation environment for traditional factory applications where an operator's hands would not normally be available for alternate input means (e.g., deploying buttons, levers etc., at a centralized control panel). The ocular tracking technology tracks movement of a user's eyes to provide input to a display. Such technology is believed to have been initially developed for use by those having a handicap or other disability that prevented use of a traditional computer keyboard or mouse.

In one embodiment of the invention, a system for providing optical input to a subcomponent of an automation system comprises an ocular tracking device configured to track eye movement of a user of the system and convert the user's eye movement to an input signal, and a PLC operatively associated with a subcomponent of an automation system. The PLC is configured to receive the input signal from the ocular tracking device and initiate implementation of a function of the subcomponent responsive to the input signal. The PLC may be connected to the ocular tracking device directly or over a network. Wireless connection can also be used. The input is either a command for the subcomponent to perform an operation, or data that is useful to an operation of the subcomponent.

The ocular tracking device can comprise a sensor for tracking the position of the user's eyes, a display screen, and, a processor configured to run a software implemented algorithm for moving a feature on the display screen in response to eye positioning information outputted by the sensor. The software can also be used to control the functioning of the sensor. The input signal provided by the ocular tracking device is responsive to placement of the feature on the display at a particular location on the display screen.

Additionally, an I/O module can be connected or otherwise associated with the subcomponent. In such instances, the input signal may be provided to the I/O module. The I/O module may then execute the command associated with the input signal (if local to the I/O module) or pass it on to the PLC.

In one example, a user of the system may look at a bin of tools within a sealed environment. Movement of the user's eyes are tracked and the PLC determines what tool to pick up (using robotics or other automation apparatus or machinery available) in the bin.

Another example of the system may involve a user inspecting articles on a conveyor belt. In this instance, the user's eye movement is tracked to determine a rate the user can comfortably inspect the articles. The PLC can adjust the rate of the motor driving the conveyor belt accordingly.

In another example, a user can utilize the system to open a door. The user can stand in a designated spot and look at a sign for opening the door. The eye movement is tracked and the PLC controlling the door can open it. Similarly a variety of operations can be performed by placing signs for such operations at different locations. This can also be used to enter a password.

The subcomponent can comprise a variety of different machines or systems. For example, the subcomponent may be a stamping machine.

In another embodiment of the invention, a system for providing hands free input to a PLC controlled subcomponent of an automation system comprises a PLC controller operatively associated with control of a machine and, an ocular tracking device configured to track the eye position of a user of the machine and provide an input signal to the PLC controller. The input signal represents a command for performing an operation of the machine or data for use by the machine. The PLC is configured to receive the input signal and convert the input signal into the command to perform an operation by the machine.

In yet another embodiment of the invention, a method for providing hands-free input for a user of a subcomponent of an automation system comprises the steps of tracking movement of the user's eyes, converting the tracking of the movement of the user's eyes into an electrical signal, relaying the electrical signal to a processor, converting the electrical signal utilizing the processor into an input signal for use by a machine, and, implementing a function of the machine responsive to the input signal.

The method can further comprise utilizing a display screen to provide feedback to the user of the tracking movement of the user's eyes, and designating a desired function to be implemented by the machine by moving a feature on the display to a first location on the display.

The input signal contains a command to perform a physical operation by the machine, or contains data useful to operation of the machine. In the latter case implementing a function of the machine responsive to the input signal can comprise storing the data by the PLC.

The present invention could be applicable to provide an ocular input as a direct input to a PLC operatively associated with a device or machine. Alternatively, the input or the electrical signal can be provided through an 10 module, or a Human Machine Interface connected to the automation system. The invention combines ocular input technology, such as that used for input to a computer, with an industrial automation environment for traditional factory applications where an operator's hands would not normally be available for alternate input means (e.g., buttons, levers etc. at a centralized control panel). The ocular input technology can be similar to the technology used for scrolling of information on a display screen using eye movement. Such technology is believed to have been initially developed for use by those having a handicap or other disability that prevented use of a traditional computer keyboard or mouse.

Other systems, methods, features, and advantages of the present invention will be, or will become, apparent to one having ordinary skill in the art upon examination of the following drawings and detailed description. It is intended that all such additional systems, methods, features, and advantages be included within this description, be within the scope of the present invention, and be protected by the accompanying claims.

### BRIEF DESCRIPTION OF THE DRAWINGS

The invention can be better understood with reference to the following drawings. The components in the drawings are not necessarily to scale, emphasis instead being placed upon clearly illustrating the principles of the present invention. Moreover, in the drawings, like reference numerals designate corresponding parts throughout the several views.

FIG. 1 is a timing chart or diagram illustrating the signal processing sequencing or flow of a system in accordance with the present invention FIG. 2 is a side diagram view of a user utilizing one embodiment of the present invention;

FIG. 3 is a front diagram view of a user utilizing another embodiment of the present invention;

FIG. 4A is a side diagram view of a user utilizing yet another embodiment of the present invention; and

FIG. 4B is a front diagram view of the embodiment of FIG. 4A.

### DETAILED DESCRIPTION

While this invention is susceptible of embodiments in many different forms, there is shown in the drawings and will herein be described in detail preferred embodiments of the invention with the understanding that the present disclosure is to be considered as an exemplification of the principles of the invention and is not intended to limit the broad aspects of the invention to the embodiments illustrated.

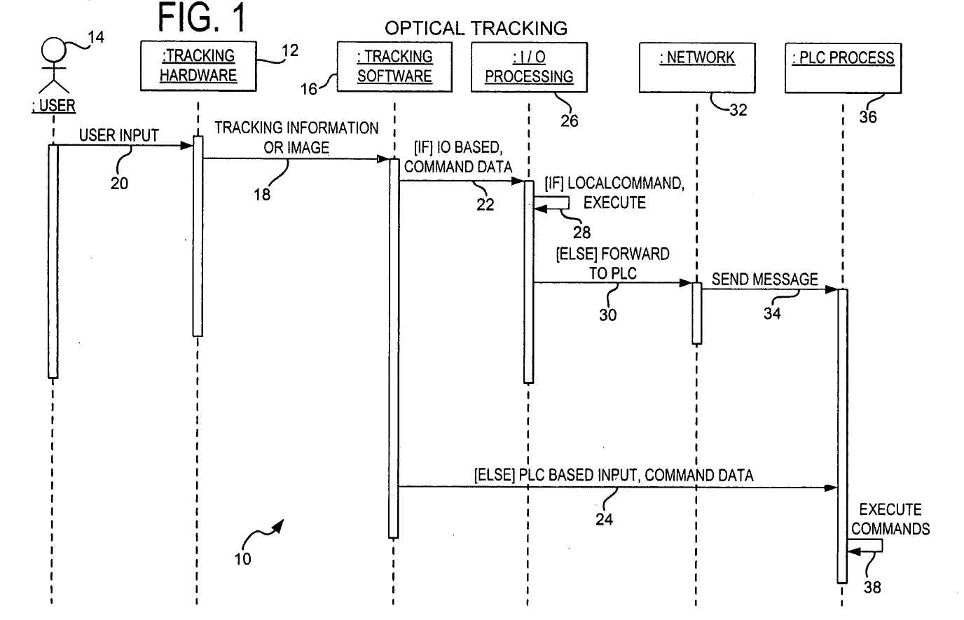

The preferred system of the present invention is utilized in connection with an automation system having a plurality of subcomponents which are controlled or monitored at least in part by programmable logic controllers (PLCs). Additionally, the subcomponents may utilize I/O modules and/or HMIs. FIG. 1 illustrates the processing steps 10 for implementing embodiments of the present invention in a networked automation system.

A subcomponent of an automation system may include one or more machines or devices controlled by a PLC. An ocular tracking device including tracking hardware components 12 is utilized with the subcomponent to provide hands free input from a user 14. The tracking hardware 12 includes a sensor which is trained on the eyes of the user 14 and tracks movement of the eyes. The tracking hardware 12 generates an electrical signal (i.e., tracking information) 18 responsive to the sensed movement 20 of the user's eyes.

The ocular tracking device also includes, or is connected to, a processor running tracking software 16 which controls movement of the sensor. The tracking software 16 also may be used for converting the electrical signals or tracking information 18 into an input signal 22 or 24 for use with the automation system. The input signal 22 or 24 is a command that: (1) directs the subcomponent to perform a particular operation and/or (2) contains data that is useful for the implementation of an operation to be performed by the subcomponent.

In one embodiment, the subcomponent may include an I/O module 26 associated with an aspect of the subcomponent. In this instance, the input signal is initially directed 22 to the I/O module for processing 26. If the input signal can be implemented locally (i.e., by the I/O module) it is executed 28. Otherwise, the input signal is forwarded 30 to a network connection 32, which in turn, forwards 34 the input signal to a PLC 36 operatively associated with controlling the subcomponent. The PLC then executes 38 the command associated with the input signal.

In another embodiment, the input signal is forwarded 24 directly to the PLC 36. Again, the PLC then executes 38 the command associated with the input signal.

The ocular tracking device associated with the subcomponent is preferably set up to allow the user 14 to stand at one or more set locations to assist or monitor operations performed by the subcomponent. The device may include a display screen that includes a feature (e.g., a cursor) that moves on the display in response to the user's eyes movement. The curser can be moved to particular locations on the display for implementing various functions.

In one example, shown in FIG. 2, an ocular tracking device 60 is connected to a PLC 62 which controls a conveyor system 64 for obtaining an item in a bin 66. A user 68 looks at a particular compartment 70 in the bin 66. The ocular tracking device 60 tracks the movement of the user's eyes and determines which compartment the user is looking at. The PLC 62 then moves the conveyor system 64 to obtain the desired item (for example, a tool for use with a particular machine in the automation system) in the bin 66.

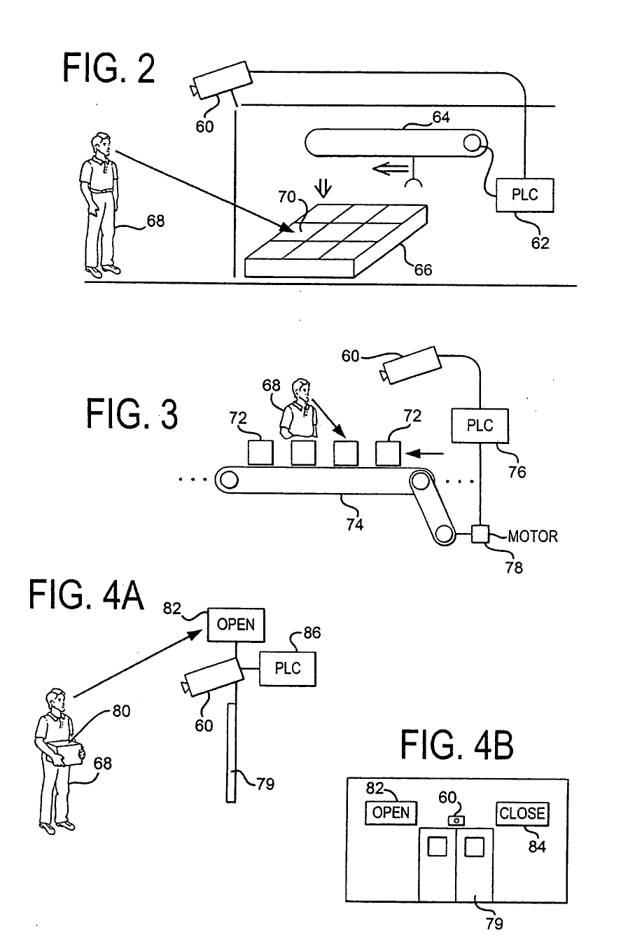

In another example shown in FIG. 3, an ocular tracking device 60 is trained on a user 68 inspecting items 72 on a conveyor belt 74. The ocular tracking device 60 monitors the user's eye movements as the user 68 inspects the items 72 and provides a signal to a PLC 76. The PLC 76 is connected to a motor 78 which operates to move the conveyor 74. The PLC 76 can control the speed of the motor 78 to move the conveyor 74 at a rate that allow the user 68 to comfortably inspect each item 72.

In yet another example of the invention shown in FIGS. 4A and 4B, an ocular tracking device 60 is mounted above an automatic door 79. A user 68 carrying a package 80 can look at an “OPEN” sign 82 positioned above the door. The ocular tracking device 60 is connected to a PLC 86 which controls operation of the automatic door 79. The ocular tracking device 60 tracks the movement of the user's eyes, and relays a signal to the PLC 86 when the user looks at the “OPEN” sign 82. The PLC 86 then opens the doors 79. In a similar manner, a “CLOSE” sign 84 can be utilized to close the door. Additionally, it is evident that this procedure can be utilized to cause a PLC to implement any number of operations (depending on what the PLC is controlling) by providing the appropriate signs positioned about the user.

It should be emphasized that the above-described embodiments of the present invention, particularly, any “preferred@ embodiments, are merely possible examples of implementations, merely setting forth for a clear understanding of the principles of the invention. Many variations and modifications may be made to the above-described embodiment(s) of the invention without substantially departing from the spirit and principles of the invention. All such modifications are intended to be included herein within the scope of this disclosure and the present invention and protected by the following claims.

1. A system for providing optical input to a subcomponent of an automation system comprising:
an ocular tracking device configured to track eye movement of a user of the system and convert the user's eye movement to an input signal;
a PLC operatively associated with a subcomponent of an automation system, the PLC configured to receive the input signal from the ocular tracking device and initiate implementation of a function of the subcomponent responsive to the input signal.

2. The system of claim 1, further comprising:
a network connecting the ocular tracking device and the PLC.

3. The system of claim 1, wherein the input signal is a command for the subcomponent to perform an operation.

4. The system of claim 1, wherein the input signal is data useful to an operation of the subcomponent.

5. The system of claim 1, wherein the ocular tracking device comprises:
a sensor for tracking the position of the user's eyes;
a display screen; and,
a processor configured to run an algorithm for moving a feature on the display screen in response to positioning information outputted by the sensor.

6. The system of claim 5, wherein the input signal provided by the ocular tracking device is responsive to placement of the feature on the display at a particular location on the display screen.

7. The system of claim 1, further comprising an IO module connected to the subcomponent.

8. The system of claim 1, wherein the subcomponent comprises a conveyor system.

9. The system of claim 1, wherein the subcomponent comprises an automatic door.

10. A system for providing hands free input to a PLC controlled subcomponent of an automation system comprising:
a PLC controller operatively associated with control of a machine; and,
an ocular tracking device configured to track the eye position of a user of the machine and provide an input signal to the PLC controller.

11. The system of claim 10, further comprising:
a network connecting the ocular tracking device to the PLC controller.

12. The system of claim 10, wherein the input signal represents a command for performing an operation of the machine.

13. The system of claim 10, wherein the input signal comprises data for use by the machine.

14. The system of claim 10, wherein the PLC is configured to receive the input signal and convert the input signal into a command to perform an operation by the machine.

15. The system of claim 10, wherein the ocular tracking device includes a processor having software for tracking the eye position of the user and converting eye position information into the input signal.

16. The system of claim 10, wherein the machine is a subcomponent of an automation system.

17. The system of claim 10, wherein the machine is a conveyor system.

18. The system of claim 10, wherein the machine is an automatic door.

19. A method for providing hands-free input for a user of a subcomponent of an automation system comprising the steps of:
tracking movement of the user's eyes;
converting the tracking of the movement of the user's eyes into an electrical signal;
relaying the electrical signal to a processor;
converting the electrical signal utilizing the processor into an input signal for use by a machine; and,
implementing a function of the machine responsive to the input signal.

20. The method of claim 19, further comprising the step of:
utilizing a display screen to provide feedback to the user of the tracking movement of the user's eyes.

21. The method of claim 20, comprising the step of:
designating a desired function to be implemented by the machine by moving a feature on the display to a first location on the display.

22. The method of claim 19, wherein the input signal contains a command to perform a physical operation by the machine.

23. The method of claim 19, wherein the input signal is provided to a PLC operatively associated with the machine.

24. The method of claim 23, wherein the input signal contains data useful to operation of the machine, and wherein implementing a function of the machine responsive to the input signal comprises storing the data by the PLC.

25. The method of claim 19, further comprising the step of:
providing the electrical signal to an IO module associated with the machine.

26. The method of claim 19, further comprising the step of:

27. The method of claim 19, wherein tracking movement of the user's eyes is performed by an ocular tracking device having a sensor for tracking the position of the user's eyes and a display for providing feedback related to movement of the user's eyes.

28. The method of claim 19, wherein the machine performs an operation relating to inspection of items on a conveyor belt.

29. The method of claim 19, wherein the machine performs an operation relating to obtaining items stored in a bin.
